(19) United States
(12) Patent Application Publication
(10) Pub. No.: US 2018/0071153 A1
(43) Pub. Date: Mar. 15, 2018

(54) ENHANCED APPEARANCE CHANGING SANITARY PAD

(71) Applicant: Deborrah R. Peoples Ellis, Miami, FL (US)

(72) Inventor: Deborrah R. Peoples Ellis, Miami, FL (US)

(21) Appl. No.: 15/702,739

(22) Filed: Sep. 12, 2017

Related U.S. Application Data
(60) Provisional application No. 62/495,387, filed on Sep. 13, 2016.

(51) Int. Cl. A61F 13/42; A61F 13/47; A61F 13/472; A61F 13/60; A61F 13/84

(52) U.S. Cl. CPC A61F 13/42; A61F 13/4704; A61F 13/47218; A61F 2013/8497; A61F 13/84; A61F 2013/422; A61F 13/60

(57) ABSTRACT

An Enhanced Sanitary Pad has various optional devices associated therewith in order to ameliorate painful conditions and also signal the amount of liquids absorbed therein. In this regard, an image is imprinted on one or more layers of a absorbent material within a sanitary pad. The image is affected by liquids such as menstrual flow to produce observable changes that signal a user to change the pad. The observable changes in the image include but are not limited to fading, color changing, glowing and or shining. Optionally, cooling crystals are embedded within an absorbent layer attached to a plastic backing material. Another option has a cooling strip associated with the pad at either end thereof, on the longitudinal sides of the pad or in appropriate locations thereabouts. Another teaching has an independent pleated protrusion having a peel strip attached thereto.

This application is related to U.S. patent application Ser. No. 15/635,154 filed on 27, Jun. 2017 herein incorporated by reference in its entirety.

### FIELD OF THE INVENTION

The present invention relates to sanitary pads for absorbing feminine discharges such as menstrual fluids and other such liquids. More particularly, the present invention relates to sanitary pads that have portions thereof that fade, change color, glow or shine as the pad becomes soiled or wet with fluids.

### BACKGROUND OF THE INVENTION

### Patent Prior Art

U.S. Pat. No. 7,192,630 to Ziltener herein incorporated by reference teaches a disk consists of one or more plies or layers (1, 2, 3) which lie one on the other and which are connected to one another in their edge region (10) and consequently have a compression region (20) on their circumference (4). Such a disk is provided for the application and/or absorption of liquid or semisolid substances. In this case, the compression region (20) is provided at a radial distance (B) from the circumference (4) of the disk. Consequently, the material fibers directed outward from the compression region (20) are so long that they are not rigid, but, instead, are slightly flexible.

U.S. Pat. No. 8,138,387 herein incorporated by reference teaches an absorbent article to be worn by a wearer having a skin-contacting sheet such as a topsheet, a backsheet, an absorbent core and a colored hydrophilic lotion, that not only helps to reduce the adherence of the menses or feces to the skin, thereby improving the ease of menses or bowl movement (BM) clean up, but that also may serve as a wetness indicator. The absorbent articles may be infant (baby) diapers, including training pants, adult incontinence articles and the like.

U.S. Pat. No. 6,596,918 herein incorporated by reference teaches a disposable absorbent article such as a training pant includes a liquid soluble wetness indicator and one or more, masking techniques for minimizing the likelihood of smudges appearing after activation. The techniques include masking graphics and peripherally disposed adhesive patterns.

U.S. Pat. No. 8,784,689 herein incorporated by reference teaches a color-changing composition and a multiple-component material including a substrate and a printed layer on the substrate. The printed layer includes a color-changing composition to indicate a change in condition, such as a change in wetness. The color-changing composition includes a matrix-forming component, a leuco dye, a Lewis Acid, a neutral surfactant and an organic solvent.

U.S. Pat. No. 7,520,873 herein incorporated by reference teaches a disposable absorbent article having an area which is visible when the article is worn. A color gradation in the area provides a coloration which varies in intensity over the area from a higher intensity of color to a lower intensity of color. A visible element, such as an indicator for indicating a change in condition of the article, is at a location where the coloration is of lower intensity or absent so as not to obscure visibility of the element.

U.S. Pat. No. 6,710,221 herein incorporated by reference teaches a disposable absorbent article such as a training pant includes active graphics that can change color during use and can provide an interactive training aid. The active graphics in particular embodiments can comprise moisture sensitive color change compositions that disappear from view or blend in with background graphics upon activation. In particular embodiments, the active graphics comprise at least two compositions that have different post-activation colors.

U.S. Pat. No. 8,637,430 herein incorporated by reference teaches a web substrate comprising an activatable colorant and at least one region comprising a topical additive. A first activated color region is produced in the web substrate upon exposure to a first external stimulus and a second activated color region is produced within the first activated color region upon exposure to a second external stimulus. The second activated color region coincides with the topical additive region.

U.S. Pat. No. 8,440,587 herein incorporated by reference teaches a method of producing a web substrate comprising an activatable colorant and at least one region comprising a topical additive. The web substrate is exposed to a first external stimulus producing a first activated color region. The first activated color region is subsequently exposed to a second external stimulus producing a second activated color region within the first activated color region. The second external stimulus is induced by application of the topical additive such that the second activated color region coincides with the topical additive region.

U.S. Pat. No. 8,871,994 herein incorporated by reference teaches a wetness sensor for an absorbent article that is formed from an ink is provided. The ink includes a proton-accepting chromogen and a proton-donating agent (or color developer). Prior to use, the ink is generally dry and in a protonated form so that it has a visible color. However, upon contact with bodily fluids (e.g., urine, fecal matter, mucus, menses, vaginal fluid, etc.), water in the fluid can lead to deprotonation of the chromogen, thereby resulting in a shift of the absorption maxima of the chromogen towards either the red (“bathochromic shift”) or blue end of the spectrum (“hypsochromic shift”). To increase the rate of the color change during use, the proton-donating agent is an aliphatic carboxylic acid that is highly soluble in the bodily fluid (e.g., urine), and therefore results in a color change that is very rapid and may be detected within a relatively short period of time.

U.S. Pat. No. 9,018,434 herein incorporated by reference teaches absorbent articles are disclosed that have been printed with active graphics alone or in combination with permanent graphics to create a wetness indicator. Different active graphic compositions and/or permanent graphic compositions are combined so as to make the wetness indicator very prominent on the article. In addition, when the absorbent article is wetted, the active graphics undergo a change that is easily noticed by the wearer or the caregiver for the wearer. In one embodiment, for instance, active graphics are positioned within a distinctive frame. The frame focuses a person's attention onto the active graphics. The active graphics fill most of the surface area of the frame such that when the absorbent article is wetted, a clearly noticeable change occurs.

U.S. Pat. No. 8,440,877 herein incorporated by reference teaches a sensing absorbent article system including an absorbent article having a liner and an outer cover, the absorbent article including a sensing means, an article design scheme, and an attachment zone having an attachment zone design scheme, wherein the attachment zone design scheme is visually differentiable from the article design scheme. Also presented is a sensing absorbent article system including an absorbent article having a liner and an outer cover, the absorbent article including a sensing means, an article design scheme, and an attachment zone adapted to indicate the proper position for a signaling device.

U.S. Pat. No. 8,933,293 herein incorporated by reference teaches a multiple layer material that includes a film layer, a moisture-reacting composition layer and an adhesive layer. The film layer has at least one printed surface. The moisture-reacting composition layer is adjacent the printed surface of the film layer. A portion of the moisture-reacting composition layer forms a framing device. The adhesive layer is adjacent the portion of the moisture-reacting composition layer forming the framing device. The multiple layer material of the invention may be used to form a component of an absorbent article having active graphics that change in appearance in response to moisture.

US Patent App. No. 2012 0330258 herein incorporated by reference teaches an article including a fibrous nonwoven layer attached to a film layer to form a laminate with an ink composition applied to a surface of the film layer which is opposite the nonwoven layer to form graphics. In use, such laminates are often positioned so that the fibrous nonwoven layer forms the visible side of the laminate with the ink composition being partially obscured by the film and nonwoven layers of the laminate. At least the surface of the film layer opposite the ink composition is treated with a softening agent which improves the visibility and clarity of the underlying graphics. Processes for forming such articles are also disclosed.

US Patent App. No. 2005 0177120 herein incorporated by reference teaches a disposable absorbent article such as a training pant includes wetness indicating graphics that provide an interactive training aid. The interactive graphics can include a permanent character graphic and one or more active object graphics, which “appear” or “disappear” from view in response to exposure to urine or the environment. The interactive graphics can also include a story line in which the permanent character graphic is illustrated performing an activity involving the active object graphic. The wetness indicating feature and the graphic story line allow the caregiver to interact with the child in an educational and motivational manner to enhance the toilet training process.

All publications, patents and patent applications referred to herein are incorporated by reference in their entirety to the same extent as if each individual publication, patent or patent application was specifically and individually indicated to be incorporated by reference in its entirety. Any information in any material (e.g., a United States patent, United States patent application, book, article) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

### Cooling Materials

Cooling materials exist in many disciplines. For example, U.S. Pat. No. 9,402,772 to Fernkvist and herein incorporated by reference; this patent teaches an absorbent article includes a breathable backsheet material layer. At least a part of the layer includes a polymeric material exhibiting a discontinuous change in free volume in response to any of the following conditions or variables: liquid contact, the presence of certain ions, temperature and/or pH. One example of such polymeric materials is side chain liquid crystalline polymers which are able to undergo a phase transition between an isotropic phase and a nematic phase. The discontinuous change in free volume involves an abrupt change of the permeability of the breathable material layer.

Further in column 7, lines 1-24 the patent continues: The Intelimer® polymer will undergo a discontinuous change of free volume at a temperature which is selected to be slightly above the wet temperature for body liquid, such as urine or blood, i.e. in the temperature range 5-50° C. depending on the environmental temperature. Above this temperature the polymer will have an isotropic (amorphous) structure in which the free volume between the polymer chains is large enough to let air and water vapour pass therethrough. When the absorbent article is wetted by body liquid and the liquid reaches the backsheet material, an evaporation process will start as the body liquid starts to evaporate and the vapours escape through the backsheet.

This will result in a cooling effect, so that the temperature in the area where the backsheet is in contact with the evaporating body liquid will be lowered to the wet temperature for body fluids like urine and blood. When reaching this temperature the polymer will undergo an abrupt discontinuous change in free volume and switch to a nematic (crystalline) phase, in which the free volume between the polymer chains is smaller and thus the permeability of the film is decreased. This will lead to a rapid and effective sealing effect reducing the risk for leakage of liquid through the backsheet. The permeability to air and vapours will of course also be reduced, but may be maintained to a certain degree.

Another type of cooling device is currently sold by Fashion Bed Group (http://fashionbedgroup.com/). This company sells various products containing polymers that it calls Crystal Gels and holds a trademark for Aere™ Crystal™ Gel Mattress & Pillow that come in various sizes. It is through the use of innovative crystal gel fibers, both the mattress and pillow protector provide a more comfortable, cooling surface. These products are woven with the rest of the material such that the crystal gel fibers cool by absorbing heat and dissipating it away from the body.

Additionally, there are various other types of cooling gels on the marketplace such as as a topical gel of methyl salicylate. Further, various over the counter gels having aloe vitamin E and other ingredients currently exist to provide cooling to areas of the body needing this treatment. Thus, there are several ways of cooling fabrics and other fibrous materials using the above innovative materials and other materials not specifically described herein.

### Vaginal Devices

Various vaginal devices exist in the marketplace that are inserted within a lady's private parts so as to absorb vaginal discharge. However, many women do not like to use the vaginal inserts because of various issues. Amongst these are discomfort, hygiene issues, religious reasons, as well as the fact that it has been shown that these devices can have toxic effects because of the various chemicals embedded therein.

### Sanitary Pads

Billions of women throughout the world experience a monthly menstrual discharge. The use of sanitary pads for absorbing feminine menstrual discharge is well known in the prior art. The primary goal of a sanitary pad is to discreetly and completely absorb the menstrual discharge without embarrassing leakage or unsightly bulges that would reveal the aforementioned. Numerous types of sanitary pads exist in the prior art and have been disclosed in various patent documents.

A sanitary pad is made of layer(s) of cellulose extracted from the walls of trees and plants associated at manufacture with layer(s) of super absorbent polymer powder derived from petroleum or other sources; alternatively, the two materials are combined into a single or multiple layer(s) of integral material. The polymer powder can absorb many times its volume and along with the cellulose is sandwiched within one or more layers of plastic backing material. If using a single plastic layer it folds back upon itself trapping the edge of the cellulose/polymer layer(s) within the plastic layer.

However, the most common manufacturing technique is for two outer layers. A first one is perforated to pass menstrual and other liquids therethrough into the absorbent cellulose and polymer; a second one does not have any perforations and attaches at its periphery with the corresponding first one so as to preserve moisture integrity within the absorbent materials sandwiched there between. Of course, adhesives, heat treatment, glues, or other types of chemical treatment keep the absorbent materials attached between the two plastic outer layers.

However, one problem with this system that has not been addressed is that users sometimes experience rashes or fail to recognize when the pad has become full. Thus, there needs to be some way to ameliorate this discomfort by helping a user know when the pad is becoming full by triggering recognition of the amount of liquids or other materials in the absorbent material contained within the confines of the pad.

Accordingly, there needs to be some solution to the aforementioned problems that ensures a liquid resistant seal at the feminine vaginal opening as well as restricting motion so as to complement the aforementioned seal.

### SUMMARY OF THE INVENTION

The present invention overcomes the deficiencies of the known art and the problems that remain unsolved by providing an enhanced appearance changing sanitary pad as described below. Also, there is an accordion style detachable option as described below.

A sanitary pad comprising:

a first plastic layer having

an absorbent material attached thereto and

a chemically alterable image imprinted on the absorbent material.

In another aspect, wherein the chemically alterable image is from a group comprising: a partially fading image, a completely fading image.

In another aspect, wherein the chemically alterable image is a color changing image.

In another aspect, wherein the chemically alterable image is a shining image.

In another aspect, wherein the chemically alterable image is a glowing image.

In another aspect, further comprising:

a raised protrusion associated with the absorbent material.

In another aspect, further comprising:

a folded surface about an external perimeter of the raised protrusion.

In another aspect, wherein the raised protrusion further comprises:

a removable raised protrusion.

In another aspect, further comprising:

a sponge associated with the raised protrusion.

A hygiene warning system comprising:

a first plastic sheet having

an absorbent layer attached thereto and

a moisture activated image associated with an absorbent layer.

In another aspect, further comprising:

an undergarment attachment integrally associated with the plastic sheet.

In another aspect, further comprising:

a raised protrusion associated with the absorbent material.

In another aspect, further comprising:

a folded surface about an external perimeter of the raised protrusion.

In another aspect, wherein the raised protrusion further comprises:

a removable raised protrusion.

In another aspect, further comprising:

a sponge associated with the raised protrusion.

An absorbent structure comprising:

a central core having

a pleated perimeter about the central core.

In another aspect, further comprising:

a base attached at an end of the central core.

In another aspect, further comprising:

a peel strip attached to the base of the central core.

In another aspect, wherein the central core further comprises:

a cylindrical central core.

These and other aspects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, in which:

FIG. 1A presents an Enhanced Sanitary Pad having an image imprinted thereon within the absorbent material in an embodiment disclosed herein.

FIG. 1B presents an Enhanced Sanitary Pad having a faded image that was imprinted as in FIG. 1A in an embodiment disclosed herein.

FIG. 2A presents an Enhanced Sanitary Pad having an image imprinted thereon within the absorbent material in an embodiment disclosed herein.

FIG. 2B presents an Enhanced Sanitary Pad having a faded image that was imprinted as in FIG. 2A in an embodiment disclosed herein.

FIG. 3A presents an Enhanced Sanitary Pad having an integral pleated protrusion (also, an accordion style detachable option) at a center portion thereof in an embodiment disclosed herein.

FIG. 3B presents an independent pleated Enhanced Sanitary Protrusion as taught in an embodiment disclosed herein.

FIG. 3C presents an independent pleated Enhanced Sanitary Protrusion having a central sponge as taught in an embodiment disclosed herein.

Like reference numerals refer to like parts throughout the several views of the drawings.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in each figure.

Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

The pads taught herein are formable in various shapes so as to improve the fit and liquid absorption. Tape and or wings are also additional options that are combinable with any of the embodiments taught herein. These pads have increased absorbability, leak prevention, enhanced comfort and obscurity. Additionally, the pads have a cooling option to alert a user when the pad is almost full.

The unique sanitary pad as taught herein has designs, patterns, or imagery that are optionally writing, drawings, clip art, pictures, press art print, or any type of pattern or printable form. The images are treated with non toxic material and or non toxic chemical substances that fade (partially or completely) or change color when soiled and or wet as an indicator that the pad is almost or full to capacity. Thus, a user easily knows that it is time to change the pad for a fresh one and the user thereby avoids having a personal embarrassment of an overflowing situation. Alternatively, the images also glow or shine thereby changing the image or even disappear completely.

Because of the simple to understand modality of fading, color changing this novel sanitary pad taught herein is a useful tool for young girls experiencing their first period or the mentally challenged. Other useful features for these demographic groups include that the pad does not have any devices designed to enter the vaginal canal. The pads optionally include wings for an extra absorbent perimeter. Another alternative has the pads attached using double sided tape to the undergarment or using dual velcro strips having hook and look conjugates one attached to the pad and one for attachment to the undergarment. The one attached to the undergarment would necessarily having an adhesive or tape side under the velcro strip side. The adhesive or tape side would have a plastic tear strip so as to reveal the tape or adhesive for attachment to the undergarment.

The sanitary pad optionally has various layers including a cotton fleece layer, a cotton wadding layer, cellulose/SAF/SAP layer and a cotton weave layer as desired. These layers are alternately plied a multiplicity of times and in varying degrees of thickness. All of this is optionally covered with another thin cotton weave or gauze layer with designs, patterns, images, writing, clip art, press art or any other images with color agents that are joined together on the inner or outer flat surface of the pad. Some may also be arranged around the edge of the pad. These pattern layers are thin and may change colors or fade when soiled almost to capacity. The imagery optionally glow or shine as appropriate.

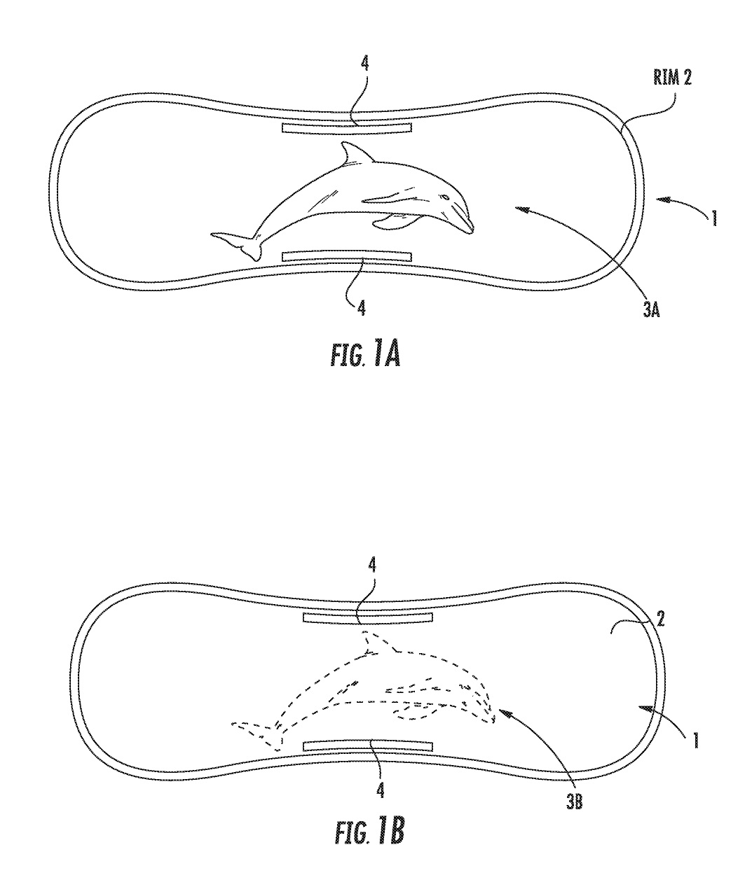

FIG. 1A presents an Enhanced Sanitary Pad having an image imprinted thereon within the absorbent material in an embodiment disclosed herein. A pad 1 has a liquid impermeable backing layer 2 of material; this backing layer 2 is preferably made from biodegradable material such as plastic and similar materials. The backing layer 2 is shown with the absorbent layer 3 attached thereto. The absorbent center 3 is made from one or more absorbent cellulose, cotton, or similar materials. This central layer captures the menstrual or other fluids that triggers the color changing, fading, glowing or shining characteristics of the material attached thereto.

The topmost layer has an image 3A such as a words, pictures, patterns, and or animals for engaging the user thereby. In this case, a dolphin is shown in the center though the particular imagery is presentable throughout any part of the absorbent layer. Additionally, the image 3A is relocatable under the topmost layer in a suitable other layer.

FIG. 1B presents an Enhanced Sanitary Pad having a faded image that was imprinted as in FIG. 1A. One or various coloring or fading chemical agents are applied to the material making up the imagery. Thus, when liquids such as menstrual fluids touch the image it fades or changes color thereby indicating to the user that it is time to change the pad.

A strip 4 of cooling material is optionally applied to a portion of the longitudinal periphery of the pad in FIG. 1A and FIG. 1B using heat treatment or adhesives. The strip 4 can also optionally have a peel strip attached to the top portion of the strip 4 using weak glues for covering the cooling material when packaged for shipment. The bottom of the cooling strip is attached to the top surface of the absorbent layer for activation via liquid association thereto. Similarly, as an option the pad has cooling crystals embedded throughout the absorbent pad for signaling the user to change her pad.

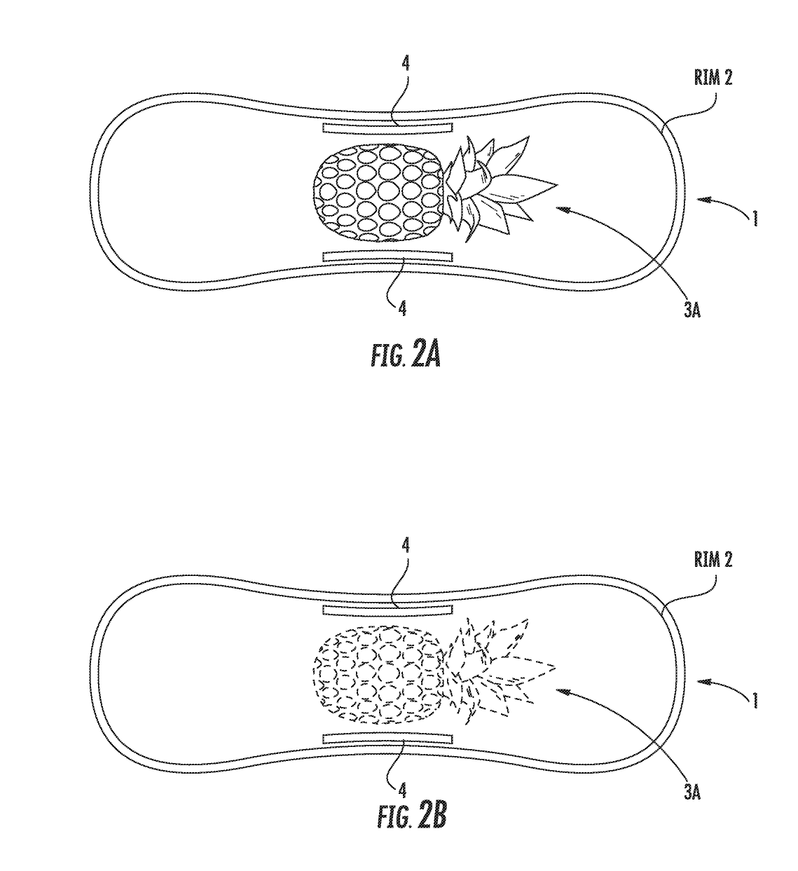

FIG. 2A presents an Enhanced Sanitary Pad having an image imprinted thereon within the absorbent material in an embodiment disclosed herein. A pad 1 has a liquid impermeable backing layer 2 of material; this backing layer 2 is preferably made from biodegradable material such as plastic and similar materials. The backing layer 2 is shown with the absorbent layer 3 attached thereto. The absorbent center 3 is made from one or more absorbent cellulose, cotton, or similar materials. This central layer captures the menstrual or other fluids that triggers the color changing, fading, glowing or shining characteristics of the material attached thereto.

The topmost layer has an image 3A such as a words, pictures, patterns, and or animals for engaging the user thereby. In this case, a pineapple is shown in the center though the particular imagery is presentable throughout any part of the absorbent layer. Additionally, the image 3A is relocatable under the topmost layer in a suitable other layer.

FIG. 2B presents an Enhanced Sanitary Pad having a faded image that was imprinted as in FIG. 1A. Various coloring or fading chemical agents are applied to the material making up the imagery. Thus, when liquids such as menstrual fluids touch the image it fades or changes color thereby indicating to the user that it is time to change the pad.

A strip 4 of cooling material is optionally applied to a portion of the longitudinal periphery of the pad in FIG. 2A and FIG. 2B using heat treatment or adhesives. The strip 4 can also optionally have a peel strip attached to the top portion of the strip 4 using weak glues for covering the cooling material when packaged for shipment. The bottom of the cooling strip is attached to the top surface of the absorbent layer for activation via liquid association thereto. Similarly, as an option the pad has cooling crystals embedded throughout the absorbent pad for signaling the user to change her pad.

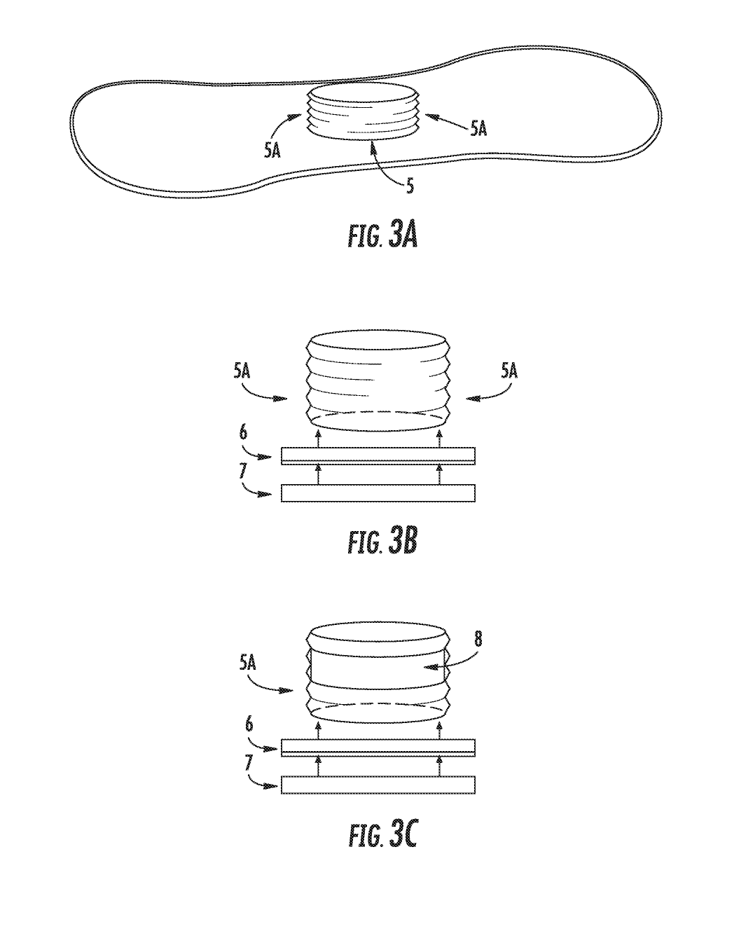

FIG. 3A presents an Enhanced Sanitary Pad having an integral pleated protrusion at a center portion thereof in an embodiment disclosed herein. An enhanced sanitary pad is shown having an integral cylindrical pleated protrusion 5 made from textile materials attached at a center portion of the pad and has a circular bottom, a circular top and pleated folds 5A arranged in a circular fashion. The protrusion 5 has a circular bottom, a circular top and pleated folds 5A. The circular bottom integrates with the lower portion of the pleated folds 5A whilst the circular top integrates with the top portion of the pleated folds 5A.

This integral pleated protrusion 5 is connected to the top layer of the enhanced sanitary pad through glues, heat treatment, adhesives, sewing or woven shaped directly from this same material forming this top layer at manufacture. The integral pleated protrusion 5 has various folds 5A roundabout the perimeter and up and down the protrusion 5 that are similar to the collapsible portion of a bellows. The material within the central portion of the cylindrical pleated protrusion 5 is less dense then the material forming the outer folds 5A so as to permit the protrusion 5 to have a vertically variable size. Alternatively, the material within the central portion of the cylindrical pleated protrusion 5 is vacant space and all flexibility that the protrusion has is dependent upon outer folds 5A. It should be further understood that the protrusion 5 is optionally removable using the adhesive strip 6 as described below in FIG. 3B, FIG. 3C so that the protrusion 5 is alternatively useable with or without the pad or solely by itself for direct attachment to an undergarment.

FIG. 3B presents an independent pleated Enhanced Sanitary Protrusion as taught in an embodiment disclosed herein. This view shows an Enhanced Sanitary Protrusion 5 that is both independent from a pad and has an adhesive strip 6 attached thereto. This adhesive strip 6 is attached on a top side thereof with strong adhesive to a circular bottom of the protrusion 5 and covers a portion or the entire circular bottom thereof. A plastic peel strip 7 at least as large as peel strip 6 attaches to week glue found on the bottom side of adhesive strip 6 so as to permit a user to peel 7 off and attach the bottom of the protrusion 5 to an undergarment.

The Enhanced Sanitary Protrusion 5 has pleated folds 5A along the sides of an integral cylindrical pleated protrusion 5 made from textile materials and has a circular bottom, a circular top and pleated folds 5A. The circular bottom integrates with the lower portion of the pleated folds 5A whilst the circular top integrates with the top portion of the pleated folds 5A. The integral pleated protrusion 5 has various folds 5A roundabout the perimeter and up and down the protrusion 5 that are similar to the collapsible portion of a bellows. The material within the central portion of the cylindrical pleated protrusion 5 is less dense then the material forming the outer folds 5A so as to permit the protrusion 5 to have a vertically variable size. Alternatively, the material within the central portion of the cylindrical pleated protrusion 5 is vacant space and all flexibility that the protrusion has is dependent upon folds 5A.

FIG. 3C presents an independent pleated Enhanced Sanitary Protrusion having a central sponge as taught in an embodiment disclosed herein. This embodiment is essentially identical to the one found in FIG. 3B with one exception. In a portion of the body of the folds 5A there is found a circular sponge 6 sewn, glued or otherwise attached to the insides of the folds 5A such that it is disposed within the center of the cylinder of the protrusion 5. This sponge 8 provides enhanced absorption as needed.

Thus, the accordion style protrusion can go up and down; it can be flattened so that the pleats of the protrusion become indistinguishable from their surroundings as they come together therewith. This happens as needed through appropriate adjustment by the user. The protrusion is made of appropriate absorbent materials or other man made materials as indicated in the disclosure.

The above-described embodiments are merely exemplary illustrations of implementations set forth for a clear understanding of the principles of the invention. Many variations, combinations, modifications or equivalents may be substituted for elements thereof without departing from the scope of the invention. Therefore, it is intended that the invention not be limited to the particular embodiments disclosed as the best mode contemplated for carrying out this invention, but that the invention will include all the embodiments falling within the scope of the appended claims.

What is claimed is:

1. A sanitary pad comprising:
a first plastic layer having
an absorbent material attached thereto and
a chemically alterable image imprinted on the absorbent material.

2. The sanitary pad of claim 1, wherein the chemically alterable image is a from a group comprising: a partially fading image, a completely fading image.

3. The sanitary pad of claim 1, wherein the chemically alterable image is a color changing image.

4. The sanitary pad of claim 1, wherein the chemically alterable image is a shining image.

5. The sanitary pad of claim 1, wherein the chemically alterable image is a glowing image.

6. The sanitary pad of claim 1, further comprising:
a raised protrusion associated with the absorbent material.

7. The sanitary pad of claim 6, further comprising:
a folded surface about an external perimeter of the raised protrusion.

8. The sanitary pad of claim 6, wherein the raised protrusion further comprises:
a removable raised protrusion.

9. The sanitary pad of claim 6, further comprising:
a sponge associated with the raised protrusion.

10. A hygiene warning system comprising:
a first plastic sheet having
an absorbent layer attached thereto and
a moisture activated image associated with an absorbent layer.

11. The hygiene warning system of claim 10, further comprising:
an undergarment attachment integrally associated with the plastic sheet.

12. The sanitary pad of claim 10, further comprising:
a raised protrusion associated with the absorbent material.

13. The sanitary pad of claim 12, further comprising:
a folded surface about an external perimeter of the raised protrusion.

14. The sanitary pad of claim 12, wherein the raised protrusion further comprises:
a removable raised protrusion.

15. The sanitary pad of claim 12, further comprising:
a sponge associated with the raised protrusion.

16. An absorbent structure comprising:
a central core having
a pleated perimeter about the central core.

17. The absorbent structure of claim 12, further comprising:
a base attached at an end of the central core.

18. The absorbent structure of claim 13, further comprising:
a peel strip attached to the base of the central core.

19. The absorbent structure of claim 13, wherein the central core further comprises:
a cylindrical central core.

20. The absorbent structure of claim 13, wherein the pleated perimeter is foldable to the point that the pleated perimeter is flattened.
